(19) United States
(12) Patent Application Publication
(10) Pub. No.: US 2023/0085475 A1
(43) Pub. Date: Mar. 16, 2023

(54) METHOD TO OBTAIN CELLS FROM LUNG TISSUE

(71) Applicant: United Therapeutics Corporation, Silver Spring, MD (US)

(72) Inventors: Benjamin Rusche, Durham, NC (US); Ashleigh Casey, Durham, NC (US); Jennifer Michaux, Raleigh, NC (US); Laila Roudsari, Durham, NC (US); Ashley R. Dockham, Raleigh, NC (US)

(73) Assignee: United Therapeutics Corporation, Silver Spring, MD (US)

(21) Appl. No.: 17/894,709

(22) Filed: Aug. 24, 2022

Related U.S. Application Data
(60) Provisional application No. 63/237,003, filed on Aug. 25, 2021.

(51) Int. Cl. C12N 5/071

(52) U.S. Cl. CPC C12N 5/0688; C12N 2511/00

(57) ABSTRACT

A method is disclosed for separating cells from a lung. Mechanical pressure can be used in one stage of the process to increase the yield of separated cells, including alveolar type II cells.

### CROSS-REFERENCE TO RELATED APPLICATIONS

This application claims priority to U.S. Provisional Application No. 63/237,003, filed Aug. 25, 2021, which is incorporated herein by reference in its entirety.

### TECHNICAL FIELD

This application relates generally to cell isolation from tissue and more particularly, but without limitation, to methods and compositions for isolating lung cells from lung tissue, as well as cells produced from such methods. These cells may be used for research, cell therapies, tissue engineering, and other applications.

### BACKGROUND

Methods to isolate different cell populations from cadaveric tissue are typically unique to each cell population, and often require different digestion enzymes, incubation times, and dissociation approaches. Due to the different isolation requirements for each cell type, tissue is often divided into separate pieces if multiple cell types are required from the same organ, thus reducing the overall possible yield for each cell type. In addition, these approaches are typically intensive and time-consuming, thereby further limiting the amount of tissue that can be processed while maintaining adequate cell health. Therefore, generating large numbers of different types of primary human cells from a single donor's tissue is a challenge. This challenge is of particular relevance to the field of tissue engineering for both autologous and allogeneic applications, where cell number requirements are high and primary cells have a limited expansion capacity. These isolation limitations could also impact personalized medicine and drug development and screening, whereby in vitro models may require generation of multicellular platforms from small pieces of donor tissue in order to achieve sufficient cellular complexity to accurately represent patient outcomes.

One specific example of a cell isolation challenge comes from processing lung tissue. The alveolar type 2 (AT2) cell is notoriously difficult to isolate. AT2 cells are often isolated from the right middle lung lobe. Isolating AT2 cells using existing methods requires multiple researchers and an entire day and yields only a few hundred million cells. This lengthy, hands-on process often limits isolation of other cell types that perform critical functions in the lung. Therefore, there is an unmet need for isolation methods that yield all critical cells of interest from donor lung tissue. Further, use of a single digestion method to isolate all cell types of interest would increase the cell yield of each cell type.

### SUMMARY

Described herein is a method to isolate different cell types using a single dissociation method from a donor organ. In some examples, disclosed is a method of isolating lung cells, such as one or more of alveolar type II cells (AT2), airway epithelial basal cells (AEP), stromal cells, and endothelial cells from human donor lung tissue. In some embodiments, the methods disclosed allow the isolation of a greater quantity of cells than other existing methods. Disclosed herein are a method of tissue dissociation and a method of cell purification, which may allow a greater number of cells to be isolated at once.

Obtaining sufficient numbers of a particular type of lung cell type, such as AT2 cells, from human donor lung tissue has been a long-standing challenge. These cells may be used to support diagnostic testing, drug discovery and development, cell therapy, or the construction of engineered organs. AT2 cells can be isolated using a method optimized in the Sannes lab at NC State (“Sannes method”, see Zhang, H., Newman, D. and Sannes, P. “HSULF-1 inhibits ERK and AKT signaling and decreases cell viability in vitro in human lung epithelial cells.” Respiratory Research. 2012; 13(1): 69), which is incorporated herein by reference in its entirety), originally developed by Leland Dobbs. AT2 isolation using the Sannes method typically yields a few hundred million AT2 cells from 1-2 lobes of human donor lung tissue. In contrast, the methods disclosed herein may enable the processing of all 5 lung lobes, which may produce as much as one billion AT2 cells without increasing staff or processing time.

Another major challenge is isolating a large number of specific types of lung cells, such as AT2 cells, with sufficient purity for downstream expansion using Sannes or other published methods. For instance, the Sannes method purification approach uses panning to remove white blood cells (differential adherence of cells to a plate), followed by a negative selection for fibroblasts. The panning approach does not easily scale and thus may be difficult with the increased number of cells generated by methods disclosed herein. Another existing purification method is use of a magnetic-activated cell sorting (MACS)- or fluorescence-activated cell sorting (FACS)-based positive selection approach based on the AT2 cell surface marker, HT2-280. However, this method is also not ideal as many fragile AT2 cells do not survive (average of 19% purification efficiency from MACS-based HT2-280 selection).

FIG. 1 shows an overview of the method of isolating specific types of cells in an embodiment directed to isolating AT2, airway epithelial basal, and stromal cells from donor lung organs. It should be noted that this is only an embodiment and similar methods should be employable to isolate other types of cells from other types of organs. Embodiments of this disclosure relate to isolating cells from biological tissue. These cells may be isolated by applying mechanical pressure to the tissue. In some embodiments, the biological tissue may be lung tissue. In some embodiments, the mechanical pressure may be applied after enzymatic digestion of the lung tissue. The biological tissue may be crushed, for instance it may be crushed in the hands of a human operator until distal tissue is liquefied. This method may enable the processing of two or more lung lobes together. This may increase the amount of material that may be processed together. The isolation method may additionally include a filtration step. The total unpurified yield post digestion and filtration may be over 30 billion cells (described herein as post-filtration sample). The isolation method may include a purification step. The final purified yield of the method may be one billion or more AT2 cells. In the case of airway epithelial basal, stromal, and endothelial cells, the method may include purification by culture selection. The culture selection may remove white blood cells.

Disclosed herein is a method of purifying cells from lung tissue. The method may include removing white blood cells. The method may include removing one or more other types of cells. In some embodiments, antibodies bound to magnetic particles are used to select for and remove the white blood cells using magnetic-activated cell sorting techniques. Antibodies bound to magnetic particles may also be used to select for and remove one or more other type of cell. The remaining cells may be alveolar type II cells (AT2). The selected cells may be one or more of airway epithelial basal cells (AEP), stromal cells, endothelial cells, among others. The method may include purifying cell populations of interest from lung tissue isolate. The method may include removing white blood cells, stromal cells, and airway epithelial basal cells. The method may include selecting for endothelial cells. The method may include removing white blood cells and AT2 cells.

In some embodiments, cell surface proteins may be used to separate cells. The method may include selecting the less sensitive cells using antibodies for at least one marker chosen from CD45, CD16, CD32, CD90, CD144, CD31, CD140b, and CD271. The method may include removing the less sensitive cells using at least one marker selected from CD45, CD90, and CD271 markers. The CD45, CD90, and CD271 beads may be used to remove white blood cells, stromal cells, and airway epithelial basal cells. In some embodiments, antibodies bound to magnetic particles are used to select for and remove the white blood cells, stromal cells, and airway epithelial basal cells. In some embodiments, a 2-step selection may be performed whereby a CD45 selection is followed by a combined CD90 and CD271 selection.

Disclosed herein is a method of forming an engineered organ. The organ may be made from a synthetic or natural lung matrix. The method may include seeding a scaffold matrix with cells obtained from a method disclosed herein. In one embodiment, an engineered lung structure may be formed by seeding a lung scaffold with cells obtained from a method disclosed herein.

Disclosed herein is an engineered organ formed by seeding a scaffold with cells obtained from a method disclosed herein. Disclosed herein is an engineered lung structure formed by seeding a lung scaffold with cells obtained from a method disclosed herein. The cells may be purified by selecting white blood cells and at least one other types of cells using antibodies for one or more cluster of differentiation (CD) markers. The CD markers may be one or more of CD45, CD16, CD32, CD31, CD90, CD144, CD140b, and CD271. In some embodiments, white blood cells, stromal cells such as fibroblasts, endothelial cells, and airway basal cells may be selected. In some embodiments, the seeded cells may be one or more of alveolar type II cells, airway epithelial basal cells, lung stromal cells, and lung endothelial cells.

As used herein, “Lung Crush Method” or “LCM” refers to a method comprising application of mechanical force to crush tissue from which cells are to be isolated. The application of force can occur during or after enzymatic digestion of the tissue. The method may comprise additional steps, and the crushing force can be applied by any suitable means, e.g., mechanical grinding or the hands of a technician.

### BRIEF DESCRIPTION OF THE DRAWINGS

FIG. 1 shows a schematic of the process of cell isolation from a biological organ using the existing Sannes lab method (top panel), comprised of use of scissors to dissociate tissue and positive selection to purify alveolar type 2 cells, compared to the method described herein (bottom panel, highlighted in yellow) comprising crushing the tissue to dissociate and negative selection to purify alveolar type 2 cells.

FIG. 2 shows the post-filtration (PF) yield per gram tissue for Sannes vs. LCM from a donor-matched comparison of the 2 tissue dissociation methods (n=3).

FIG. 3 shows post-filtration (PF) AT2 purity (HT2-280+%) for Sannes vs. LCM from a donor-matched comparison of the 2 dissociation methods (n=3).

FIG. 4 shows the theoretical AT2/g tissue for Sannes vs. LCM from a donor-matched comparison of the 2 dissociation methods (n=3). Theoretical AT2/g is calculated as total cells/g multiplied by HT2-280%.

FIG. 5 shows the post-selection AT2 purity (HT2-280%) of CD45/CD90/CD271 depleted AT2 samples for Sannes vs. LCM from a donor-matched comparison of the 2 dissociation methods (n=3).

FIG. 6 shows the post-selection AT2 per gram tissue for Sannes vs. LCM from a donor-matched comparison of the 2 dissociation methods (n=3).

FIG. 7 shows the average AT2 yield for each isolation method from a donor-matched comparison of the 2 dissociation methods (n=3).

FIG. 8 shows the selection efficiency comparison between Sannes vs. LCM calculated based on the actual AT2 Yield after purification divided by the theoretical AT2 yield prior to purification.

FIG. 9 shows a summary of AT2 cell isolation improvements when the LCM process was scaled to utilize all of the lung tissue from a single donor.

FIG. 10 shows a schematic of the process, highlighting one method to digest and dissociate all of the lung tissue, and the purification methods that could be used to separate airway epithelial basal cells, stromal cells, AT2 cells, and endothelial cells from lung tissue.

FIG. 11 shows all 4 cell types isolated from 1 donor according to an embodiment.

FIG. 12 shows images of the lung crush method.

FIG. 13 shows a summary of cell yield and purity from lungs where lung crush method was used to isolate 4 different cell types from one donor (airway epithelial basal cells, stromal cells, AT2 cells, and endothelial cells).

FIG. 14 shows an example of airway epithelial basal cells that were obtained from the lung crush method.

FIG. 15 shows an example of stromal cells that were obtained from the lung crush method.

FIG. 16 shows an example of endothelial cells that were obtained from the lung crush method.

FIG. 17 shows a schematic of one embodiment of the process to obtain airway epithelial basal cells, stromal cells, AT2 cells, and endothelial cells from lung tissue using a different purification method compared to what was described in FIG. 10.

FIG. 18 shows an AT2 isolation and characterization summary from the lung crush method and the negative selection strategy described herein.

### DETAILED DESCRIPTION

Cells isolated from human or animal organs may be used to support in vitro diagnostic and pharmaceutical testing, cell therapy development, and cellularization of scaffolds for regenerative medicine. These cellularized scaffolds may be used for transplantation into patients as clinical products. Obtaining sufficient numbers of a particular lung cell type, however, has been a long-standing challenge in the field. One such example is the isolation of AT2 cells from human donor lung tissue to use in the formation of an engineered lung tissue. The isolated AT2 cells may be banked, expanded, and used to cellularize porcine or 3D-printed lung scaffolds. These porcine or 3D-printed lung scaffolds may be transplanted into patients. The isolated AT2 cells may also be used to support research of AT2 cell identity and function, growth characteristics, disease states, and drug candidate screening in different platforms.

Isolations of AT2 cells have been performed using a method developed in the laboratory of Philip Sannes at NC State (herein described as the Sannes method). AT2 isolation using the Sannes method typically yields a few hundred million AT2 cells from 1-2 lobes of human donor lung tissue. Thus, the Sannes method must be repeated multiple times on different donors to secure a billion or more cells, leading to a high cost in time and material. Furthermore, pooling of cells from different donors for human cells, tissues, and cellular and tissue-based products is restricted by the FDA.

In contrast, the methods disclosed herein may enable the processing of all five lung lobes, which may produce as much as 1 billion AT2 cells, without increasing staff or processing time. Additionally, it may allow the processing of larger amounts of cells that may allow large-scale expansion in bioreactors. The methods disclosed herein allow for the production of an increased number of cells, which may decrease the need to isolate cells from additional donors. It may also allow for the build of donor-matched banks of multiple lung cell types and allow the repopulation of scaffolds with cells derived from a single donor. This is an important consideration for allogeneic tissue products, whereby using cells from a single donor with a close HLA match may be important to prevent organ rejection.

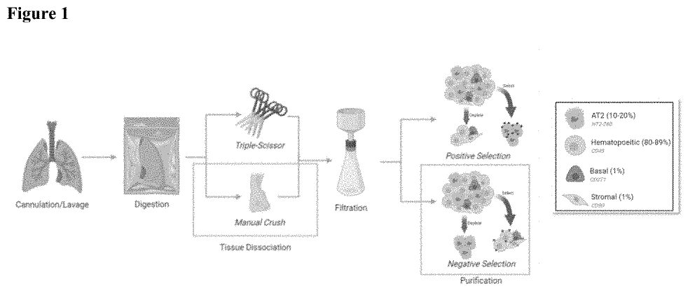

Additionally, it has been difficult to isolate a large number of AT2 cells or other specific lung cell types with sufficient purity for downstream expansion using Sannes or other published methods. Disclosed herein is a purification strategy that resolves this challenge. This purification method uses negative selection to remove non-AT2 cells that may overgrow in downstream cultures, leaving the sensitive AT2 cells unlabeled and in the negative population for downstream use. The positively selected non-AT2 cells can be seeded into culture to generate banks of other cell types of interest. FIG. 1 shows an overview of the method of isolating specific types of cells in an embodiment directed to maximizing the number of isolated AT2 cells from donor lung organs with sufficient purity for downstream culture and expansion. It should be noted that this is only an embodiment and similar methods should be employable to isolate other types of cells from other types of organs.

FIG. 1 shows a schematic of one embodiment of a method of cell selection from a biological organ. In this embodiment, the biological organ is a lung and the desired purified cell is an AT2 cell. A lung is secured from a donor. The lung is cleaned, such as by lavaging the airway with a buffer solution. The lung is then digested with enzymes, such as elastase or collagenase. At this point, the cell tissue can be dissociated either by the Sannes method, or the Lung Crush Method.

The Sannes Method (SM) may include removing the large, white airways and large chunks of undigested tissue. Small pieces of digested tissue may be transferred to a cup, minced using three pairs of surgical scissors taped together (referred to as triple-scissor) and collected. This process may be repeated several times until all of the digested tissue is minced.

The Lung Crush Method (LCM) may involve crushing the entirety of the digested tissue all at once using an object such as a hand or a mechanically automated crushing device, such as one using a rollers in series or parallel to apply force to crush the tissue. Crushing may include tearing open the pleura and allowing the digested tissue and cells to pour out and collect in a receptacle. Crushing may include squeezing the digested tissue. Crushing may include pulling apart the tissue. Crushing may include wringing out the tissue to collect additional cell suspension. The lung tissue may be crushed until only the airways and pleura remains. The airway tissue may be removed and the crushed tissue collected. The cell suspension following crushing has minimal undigested pieces of tissue remaining. In contrast, after cutting up the tissue using the Sannes method, pieces of tissue ranging from ˜1 to 5 mm are visible throughout the cell suspension. For example, tissue processed using the LCM may have no more than 20%, 10%, 5%, 2%, or 1% by weight of tissue pieces that are 1 mm, 2 mm, or 5 mm or more in diameter. In some embodiments, tissue processed using the LCM contains no more than 5% by weight of tissue pieces that are 5 mm or more in diameter. In some embodiments, tissue processed using the LCM contains no more than 5% by weight of tissue pieces that are 2 mm or more in diameter. In some embodiments, tissue processed using the LCM contains no more than 5% by weight of tissue pieces that are 1 mm or more in diameter. Determining the relative amount of pieces of tissue of a particular size can be accomplished using sieves, mesh, or the like of the appropriate size.

After being dissociated, the collected liquid may be filtered. The liquid may be filtered through surgical gauze or a mesh, silk, or nylon filter. The liquid may be filtered multiple times and through multiple filters. The liquid may also be centrifuged one or more times and the cell pellet resuspended.

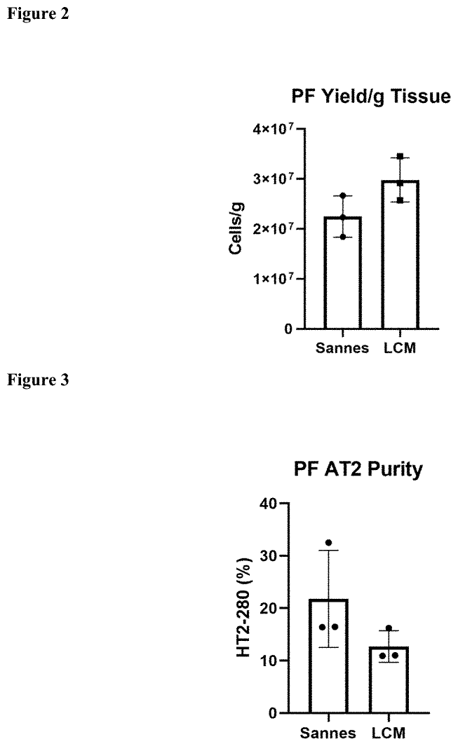

Three head-to-head isolations were performed on donor-matched tissue to compare the LCM to the Sannes method prior to scaling up the LCM. For each donor in this comparison study, the tissue was divided into left and right lungs. One lung from each donor was processed using the Sannes method and one was processed using LCM. The lung that was assigned to each process was changed with each donor, as well as the operator performing the isolation. Data from this comparison study are included in FIGS. 2-4.

FIG. 2 shows the total post-filtration (PF) cell yield per gram tissue for Sannes vs. LCM (n=3). These data demonstrate that the yield per gram of tissue is similar for the Sannes and the Lung Crush Method. There is no statistically significant difference in the amount of cells purified per gram of tissue (Welch's t test, p<0.05).

FIG. 3 shows the post-filtration (PF) AT2 cell purity for Sannes vs. LCM (n=3) following dissociation. There was no significant difference in the purity of the cells post-filtration between the two dissociation methods (Welch's t test, p<0.05).

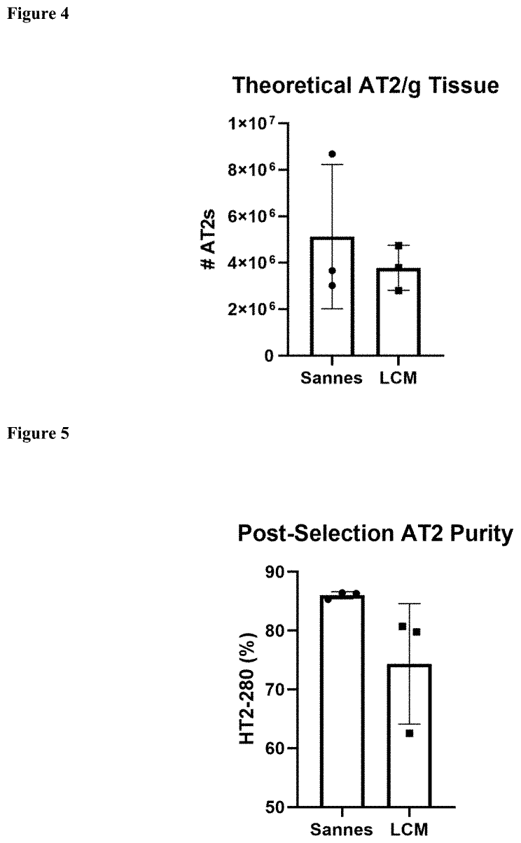

FIG. 4 shows the theoretical number of AT2 cells per gram tissue for Sannes vs. LCM (n=3), calculated as the post-filtration yield multiplied by the post-filtration purity. No statistically significant difference was evident in the theoretical yield of AT2 cells isolated from tissue using the two different methods of dissociation (Welch's t test, p<0.05).

After filtration, the desired cells may be purified. The selection process may be a negative or positive selection process. In the Sannes purification method, undesired cells may be removed by differential adherence to non-tissue-culture Petri dishes with or without the use of antibodies. In some embodiments, a combination of differential adherence and magnetic removal may be used. The Sannes AT2 purification process may involve plating and panning to remove white blood cells and stromal cells, such as fibroblasts. The purification process may involve using an antibody, such as an AS02 antibody to selectively attach to the stromal cells. The antibody may be attached to a metal particle, allowing the stromal cells to be removed magnetically. Another commonly used method is to positively select the cells using an antibody for the AT2 cell surface marker, HT2-280 (Terrace Biotech, Mouse IgM monoclonal antibody), followed up by staining with an anti-mouse IgM magnetic bead. While positive selection via HT2-280 results in a high purity sample with low levels of contaminating cell types that may overgrow the culture, the purification efficiency is low with this selection method, thus leading to a low overall AT2 yield.

In the purification method described herein, other non-AT2 cells may also be removed. For instance, CD45, CD90, and CD271 antibodies may be added to bind to white blood cells, stromal cells, and airway basal cells. These antibodies may be bound to a metal particles. The metal particle, antibodies, and attached blood cells, stromal cells, and airway basal cells may be removed magnetically.

The antibodies bound to magnetic particles may also be used to select for and remove one or more types of cells to leave the most sensitive, desired cells behind. The desired cells left behind in the negative population may be banked for future use. The isolated cells may be alveolar type II cells (AT2). The isolated cells may be one or more of airway epithelial basal cells, stromal cells, endothelial cells, among others.

Depending on the identity of the desired cell, the method may include removing white blood cells, stromal cells, and/or airway epithelial basal cells. The method may include removing white blood cells and/or alveolar type II cells. Magnetic beads bound to antibodies for cell surface proteins may be used to selectively separate the cells that are not AT2 cells. The selected cells may be removed using at least one antibody for a cell surface protein selected from CD45, CD16, CD32, CD90, CD31, CD144, CD140b and CD271. The selected cells may be removed using at least one antibody for a cell surface protein selected from CD45, CD90, and CD271 markers. The CD45, CD90, and CD271 antibodies may be used to white blood cells, stromal cells, and airway basal cells from the AT2 population. Alternate markers may be used to remove all but the desired cell from the sample.

A negative selection approach using magnetic beads bound to antibodies for CD45, CD90, and CD271 was performed to purify AT2 cells from the head-to-head isolation tests of LCM and Sannes dissociation methods that were presented in FIGS. 2-4. Post-purification data from these comparisons are included in FIGS. 5-8.

FIG. 5 shows the post-purification AT2 purity (% of cells positive for HT2-280) of CD45/CD90/CD271 depleted samples from Sannes vs. LCM comparisons. No statistically significant difference was found between the two samples (n=3 donors, Welch's t test, p<0.05).

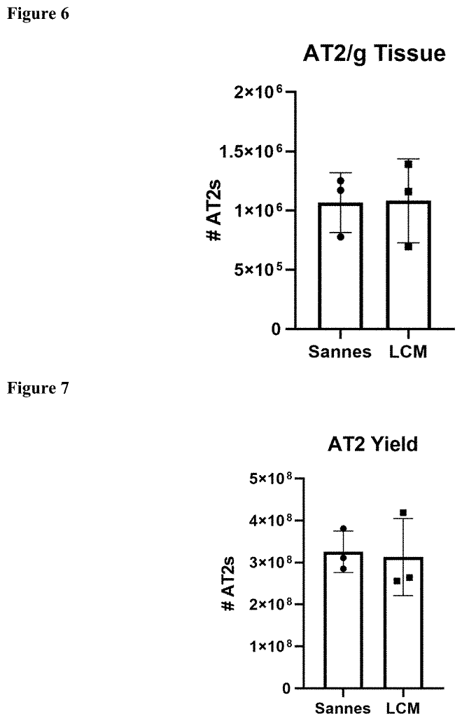

FIG. 6 shows the post-purification AT2 cell number per gram of tissue from Sannes vs. LCM comparisons. No statistically significant difference was found between the two samples (n=3 donors, Welch's t test, p<0.05).

FIG. 7 shows the average total post-purification AT2 cell yield for each of the Sannes and LCM isolation methods performed on similar amounts of tissue (donor 1: 400 g Sannes, 380 g LCM; donor 2: 244 g Sannes, 220 g LCM; donor 3: 306 g Sannes, 301 g LCM). No statistically significant difference was seen between the two samples, as expected given that the same amount of tissue was processed and the same purification strategy was performed (n=3 donors, Welch's t test, p<0.05).

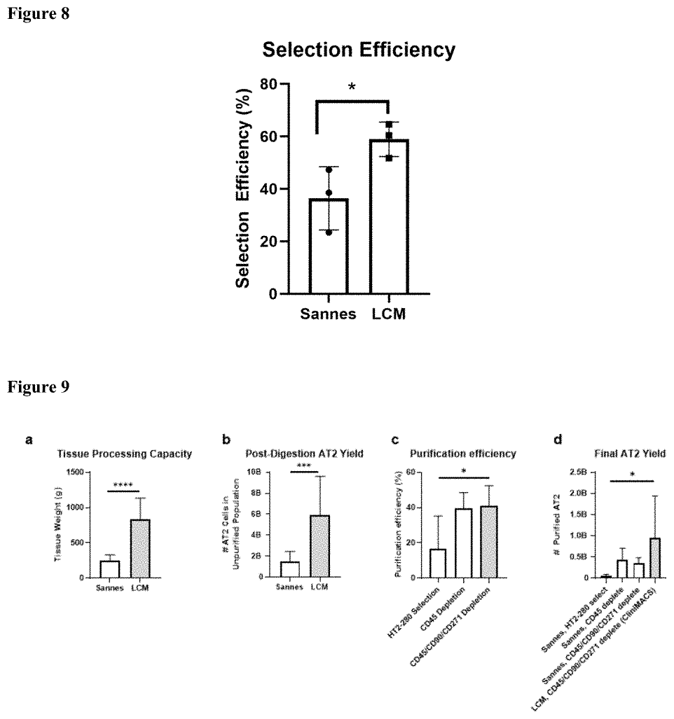

FIG. 8 shows the selection efficiency comparison between Sannes vs. LCM, calculated based on AT2 yield after purification divided by the theoretical AT2 yield prior to purification. The Lung Crush Method yielded a statistically significant improvement in selection efficiency when compared with the Sannes Method (n=3 donors, Welch's t test, p<0.05).

Following the head-to-head comparisons, the LCM was scaled up to process both the left and right lung tissue (bilateral lungs) from donors. The triple scissor dissociation step for the Sannes protocol is labor- and time-intensive, which limits the amount of tissue that can be processed at one time. The simplified dissociation with lung crush method allows the entirety of the lung tissue from one donor to be handled at once and decreases the overall processing time.

FIG. 9 shows a summary of the improvements seen in one embodiment of the methods disclosed herein. In this embodiment, AT2 cells were isolated from donor lung tissue using the scaled up Lung Crush Method compared to historic Sannes method (inclusive of triple scissor minced samples) data. The AT2 cells were then purified using the negative magnetic bead selection method and compared to historic data using other established purification methods. The Lung Crush Method dissociation approach significantly increased (a) tissue processing capacity and (b) the number unpurified AT2 cells in the post-digestion sample (theoretical AT2 cell yield). This dataset included all full-scale LCM runs, where bilateral lungs (all lung lobes) were processed. (c) Different purification strategies were compared on cells isolated from the Sannes triple scissor method. The CD45 depletion method trended towards improving the purification efficiency compared to the HT2-280 selection method. However, while the HT2-280 selection method produced cells with sufficient purity for downstream AT2 culture, the CD45 depletion on its own did not. Thus, the CD45/CD90/CD271 depletion method was established and uses negative selection by surface markers to deplete basal (CD271+) and fibroblasts (CD90+) with CD45+ cells. (d) A comparison of the final AT2 yield generated from different isolation and purification approaches. The average AT2 yield per donor was 73M using the original method (Sannes, HT2-280 selection performed on a small-scale MACS instrument). The Sannes method combined with the CD45 depletion or the CD45/CD90/CD271 depletion trended towards an increase in AT2 yields. However, the lung crush method combined with CD45/CD90/CD271 resulted in the highest AT2 yield. In addition, given the higher cell yields, the large-scale CliniMACS purification instrument was required for full-scale processing. This dataset includes full-scale runs of LCM, where bilateral lungs were processed and purified on the CliniMACS using depletion tubing where no process errors or deviations occurred. These new methods combined demonstrated a statistically significant increase to an average of 930 million AT2 cells isolated from 1 donor. (t-test for a and b, ANOVA, Tukey's multiple comparisons test for c and d, *p<0.05, ***p<0.001, ****p<0.0001).

These purification methods are summarized in the following tables:

HT2-280 Selection Staining

| Staining Step | Concentration | Protocol |
|---|---|---|
| HT2-280 antibody | 30 μL antibody/ | Spike in antibody to cell suspension |
| incubation | mL cell suspension | Incubate for 30 min at 4° C. |
| | | Dilute by adding DMEM/F12 |
| | | medium + 100 U/mL DNase at 6x |
| | | the volume of the staining solution |
| | | Centrifuge at 300 x g for 5 min |
| Resuspension | 80 μL/1.0 × 10⁷ | Resuspend cells in DMEM/F12 |
| | cells | medium + 100 U/mL Dnase |
| Anti-Mouse IgM | 20 μL/1.0 × 10⁷ | Spike in microbeads |
| Microbeads | cells | Incubate for 15 min at 4° C. |
| | | Dilute by adding DMEM/F12 |
| | | medium + 100 U/mL Dnase at 6x the |
| | | volume of the staining solution |
| | | Centrifuge at 300 x g for 5 min |
| Final Resuspension | 500 μL/1.0 × 10⁷ | Resuspend cells in DMEM/F12 |
| | cells | medium + 100 U/mL Dnase |

CD45, CD271 and CD90 Depletion Staining

| Staining Step | Concentration | Protocol |
|---|---|---|
| Resuspension | 40 μL/1.0 × 10⁷ | Resuspend cells in DMEM/F12 |
| Volume | cells | medium + 100 U/mL DNase |
| Bead incubation | 20 μL of each bead | Spike in microbeads |
| (CD45, CD271, | type/1.0 × 10⁷ | Incubate for 45 min at room |
| CD90) | cells | temperature |
| | | Dilute by adding DMEM/F12 |
| | | medium + 100 U/mL DNase at 10x |
| | | the volume of the staining solution |
| | | Centrifuge 300 x g for 5 min |
| Final Resuspension | 1.25 × 10⁸ mL | Resuspend cells in DMEM/F12 |
| | (CliniMACS) | medium + 100 U/mL DNase |

### EXAMPLES

The following example describes specific aspects of some embodiments of this disclosure to illustrate and provide a description for those of ordinary skill in the art. The examples should not be construed as limiting this disclosure, as the example merely provides specific methodology useful in understanding and practicing some embodiments of this disclosure.

### Comparative Example 1: Dissociation and Purification Using Sannes Methods

In this method, a lobe (usually the right middle), is dissected out for processing.

Cleaning: The lung lobe vasculature was perfused free of blood with Solution II (an aqueous solution of NaCl, Na₂HPO₄, HEPES, CaCl₂), and MgSO₄ 7 H₂O) at 37° C. Air was removed from the lung lobe, the lobe was cannulated, and washed with Solution I (an aqueous solution of NaCl, Na₂HPO₄, HEPES, glucose and EGTA). Lavage was repeated until draining solution ran clear.

Digestion: Elastase was dissolved in Solution II at 37° C. The lung lobe was incubated in a water bath set to 37° C. and filled with the warm elastase solution. The lung was allowed to digest until it became well-relaxed.

Tissue Dissociation (Sannes Method): Large, undigested chunks of tissue were excised. Large white airways were removed and discarded. Smaller lobe pieces were added to a chilled cup on ice containing 5 mL cold DNase solution (25 mg DNase in 50 mL of Solution II). These smaller lobe pieces were minced in batches with three pairs of surgical scissors “triple scissors” held or taped together in tandem. The minced cell solutions were collected into a 1 liter flask kept cold on ice. Once all of the tissue was processed, FBS was added to the cell suspension and the flask was shaken vigorously in a water bath (37° C.) for 3 minutes.

Filtration: The cell suspension was filtered through a layer of moistened surgical gauze up to 3 times. The cell suspension was filtered through 2 layers of moistened surgical gauze. This was repeated at least once to remove most of the large tissue pieces. The cell suspension was then filtered once or twice through moistened triple layer gauze. The cell suspension was filtered through a 165 μm silk or nylon mesh.

Centrifugation: The cell suspension was centrifuged at 200×g for 10 minutes at 4° C. The supernatant was discarded and the cell pellet resuspended in 5 mL DMEM media.

Plating: A petri dish was prepared with 500 μg/mL human IgG in Tris buffer at pH 9.5. In some cases, the dishes were incubated overnight at 4° C. About 5 mL of cell solution was delivered to the prepared IgG dishes.

Panning: The prepared cell dishes were panned for up to one hour in the incubator and until the white blood cells appeared well-adhered and gray but AT2 cells were still refractile and not attached. Fibroblasts also began to attach. The cell dishes were removed from the incubator and gently rocked to mobilize the AT2 cells. The unattached cell solution was collected and centrifuged at 200×g for 10 minutes at 4° C. The supernatant was discarded.

Fibroblast depletion option 1-Differential Adherence: The fibroblast population was depleted by differential adherence to non-tissue culture-treated Petri dishes for ˜1 hour.

Fibroblast depletion option 2-Magnetic Removal: The fibroblast population was depleted using an AS02 anti-fibroblast antibody negative selection step. The cell pellet was resuspended in DMEM. AS02 antibody was used to selectively attach to the fibroblasts. Tubes of cells and antibodies were gently rolled for a 10 minute incubation period at 4° C. DMEM/0.1% cell culture-grade BSA was added and the solution was centrifuged (10 min, 800 rpm, 4° C.). The supernatant was removed and the cells were resuspended in DMEM/0.1% BSA.

Dynabead prep: Pan-mouse IgG dynabeads were washed in 1 mL DMEM/0.1% BSA, collected magnetically and resuspended in DMEM/0.1% BSA. The Dynabeads were added to the cells and the solution incubated for 30 min at 4° C., rolling slowly end-over-end. The fibroblasts were removed magnetically for ˜2 minutes by a DynaMag-15 magnet. The unattached AT2 cells were poured off to collect and count. The cells were centrifuged to concentrate and to exchange medium for seeding. The pellets were resuspended in DMEM with 10% FBS and 2× antibiotic/antimycotic. The cells were counted and stored for future use.

Fibroblast depletion option 3: Fibroblasts were depleted using a combined method of option 1 and option 2.

### Example 2: AT2, Airway Epithelial Basal, and Stromal Isolation Using Lung Crush Method and CD45/CD90/CD271 Depletion

In this method, bilateral lungs (all lung lobes) are used for processing.

Cleaning: The lung airway was cannulated and instilled with 1 L HBSS (−MgCl₂, −CaCl₂)). The HBSS was drained from the lung with gentle massage. Lavage was repeated 3 times. 2 final rinses were completed with HBSS (+MgCl₂, +CaCl₂)).

Digestion: Elastase, collagenase type IV, calcium chloride, and DNase was dissolved in HBSS (−MgCl₂, −CaCl₂) at 37° C. and instilled into the lungs (collagenase type IV and Dnase are not used in the Comparative Example 1). The lungs were placed into a Whirlpak bag and placed in a water bath set at 37° C. and the lungs were allowed to digest for approximately 45 minutes.

Tissue Dissociation (Lung Crush Method): Wearing sterile gloves, a human operator placed a hand inside bag. The pleura was torn open and the lung tissue was pulled apart and crushed by hand until only the airways remained. At this point, the remaining airway tissue was removed from the bag and disposed. The liquid contents of the Whirlpak bag were collected.

Filtration: The cell suspension was filtered through a series of mesh sheets with decreasing pore size (2000 μm, 1000 μm, 200 μm, 100 μm). After filtering, the cell suspension was brought up in DMEM/F12 media with DNase. 5% FBS was added to the cell suspension and mixed.

Centrifugation: The cell suspension was centrifuged at 300×g for 8 min. The supernatant was discarded and the cell pellet was resuspended in 5 mL DMEM/F12 with DNase.

Once a cell suspension was secured according to the above methods, AT2 cells, AEP cells, and stromal cells were purified according to the following method.

Magnetic Bead labeling of Stromal Cells, Airway Basal Cells, and White Blood cells: The cells were counted, for instance, with a K2 Cellometer (Nexcelom). The cell suspension was centrifuged, for instance at 300×g for 5 minutes at 4° C. The cell suspension was resuspended in media. In some cases, the media included DNase. CD45, CD90 and CD271 beads were added to bind with white blood cells, fibroblasts, and airway basal cells, respectively. In some embodiments, the beads were added in excess to the number of stromal cells, airway basal cells, and/or white blood cells expected in the sample. The sample was mixed well and incubated. In some embodiments, incubation occurred for 45 minutes at room temperature. The cells were washed, centrifuged, and resuspended in media.

Magnetic Separation of AT2 cells from Stromal Cells, Airway Epithelial Basal Cells, White Blood cells: The cells were placed into a container, such as a blood transfer bag, and attached to CliniMACS tubing set. A depletion program was selected, in this example specifically the Depletion program 3.1 program on the CliniMACS™ system (a cell purification system). The cells (AT2 cells) that were not selected using the Depletion program were counted via the K2 Cellometer (cell counter) and stored for future use.

The cells that were selected (CD45+/CD90+/CD271+) were split and seeded at 300,000-400,000 cells/cm² into separate flasks in culture medium designed to support airway epithelial basal or stromal cells. These cultures generated purified populations of AEP and stromal cells over passage.

### Example 3: Stromal Purification by Culture Selection Following Lung Crush Method Tissue Dissociation

Lung cells were isolated from lung tissue according to the following method. The donor lung tissue was cleaned and digested according to the methods disclosed herein or known to those skilled in the art. The lung tissue was dissociated using a method such as the Lung Crush Method. The cell suspension was filtered through surgical gauze, nylon, mesh, or other porous material according to methods disclosed herein or other methods known in the art.

Once a cell suspension was secured according to the above methods, stromal cells were purified according to the following method.

A post-filtration sample was frozen down. A sample taken following isolation was evaluated for CD90 expression. Post-filtration cells were thawed and seeded in stromal cell medium at a concentration of 3,000 CD90+ cells/cm². These cultures generated a purified population of stromal cells over passage. This purification method serves as an alternative to the use of selected cells to generate a stromal cell culture, and enables maximization of the selected population to be used for generation of airway epithelial basal cell cultures.

### Example 4: Endothelial Cell Isolation by Positive Selection Following Lung Crush Method

Endothelial cells were isolated from lung tissue according to the following method. The donor lung tissue was cleaned and digested according to the methods disclosed herein or known to those skilled in the art. The lung tissue was dissociated using a method such as the Lung Crush Method. The cell suspension was filtered through surgical gauze, nylon, mesh, or other porous material according to methods disclosed herein or other methods known in the art.

Once a cell suspension was secured according to the above method, endothelial cells were selected according to the following method.

Magnetic Bead labeling and Separation of Endothelial Cells: The cells were counted, for instance, with a K2 Cellometer (Nexcelom). The cell suspension was centrifuged, for instance at 300×g for 5 minutes. The cell suspension was resuspended in media. In some cases, the media contained DNase. CD45 beads were added to the cell suspension to bind with white blood cells. In some embodiments, incubation occurred for 15 minutes. The cells were placed into a container, such as a blood transfer bag, and attached to CliniMACS tubing set. A depletion program was utilized to select for the CD45 positive white blood cells. CD31 beads were then added to the negative fraction from the first purification step, in order to bind with endothelial cells. In some embodiments, the beads were added in excess to the number of endothelial cells expected in the sample. The sample was mixed well and incubated. In some embodiments, incubation occurred for 15 minutes at room temperature. The cells were washed, centrifuged, and resuspended in media. A MultiMACS instrument was used to then select for the endothelial cells. The endothelial cells that were selected were counted via the K2 Cellometer and seeded into culture in endothelial cell culture medium.

Endothelial cells can also be obtained by seeding cells directly into culture following digestion with a purification using CD31 selection following 1-2 passages of culture.

### Example 5—Isolation of 4 Cell Types from 1 Donor

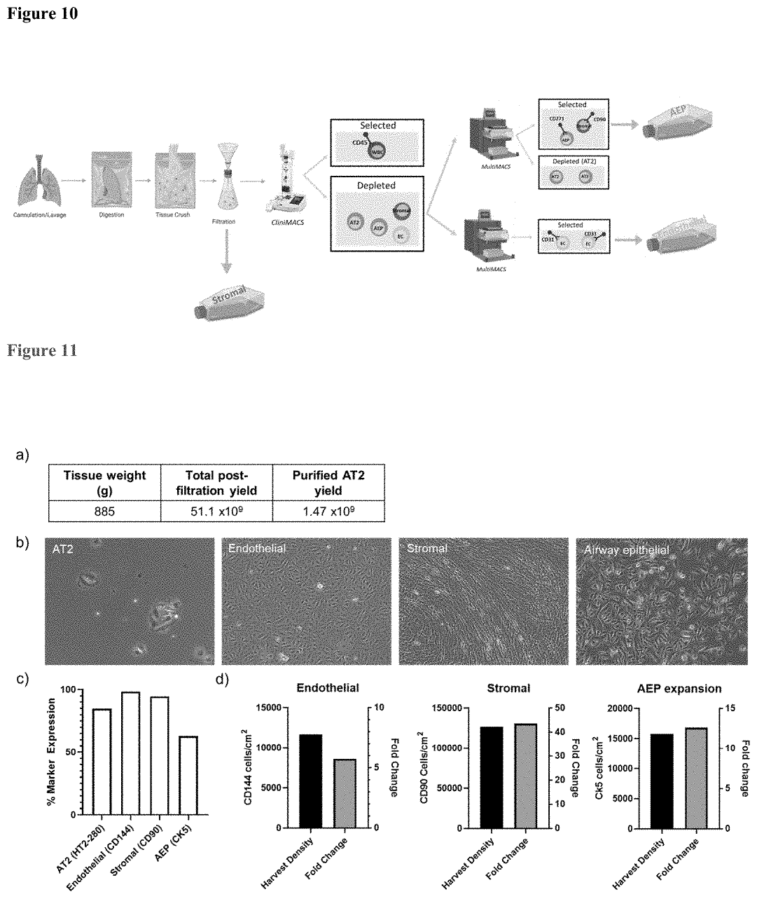

FIG. 10 shows a schematic of an embodiment of the process, highlighting a method that was used to digest and dissociate all of the lung tissue, and the purification methods that were used to separate airway epithelial basal cells, stromal cells, AT2 cells, and endothelial cells from lung tissue. All lung lobes were digested and dissociated using the lung crush method. To generate a purified population of stromal cells, the post-filtration sample was seeded directly in stromal media and grown for 3 passages (P2). 20B post-filtration cells were stained with CD45 beads and purified using the Depletion 3.1 program on the CliniMACS. The depleted sample from this first purification was then stained with CD90 and CD271 and purified on a MultiMACS instrument. The depleted cell sample from the second purification step was designated as the AT2 population. The selected cell sample from the second purification step was seeded into culture in airway epithelial basal cell growth media and further purified via culture for 3 passages (P2). To generate endothelial cells, a separate aliquot of post-filtration sample was stained with CD45 beads first and depleted on the CliniMACS, followed by CD31 beads to purify via positive selection on a MultiMACS instrument. The selected sample was seeded into culture in endothelial cell media and grown for 4 passages (P3).

FIG. 11 shows the results of the isolation of 4 lung cell types (AT2, endothelial, stromal, and airway epithelial basal) from 1 donor described in FIG. 10. FIG. 11 (a) shows tissue isolation (weight and total post-filtration cell yield) and total AT2 yield information, and FIG. 11 (b) shows morphology images of each of the 4 cell types, AT2: 24 hrs after seeding, 20× objective; endothelial: passage 3, 10× objective; stromal and airway epithelial: passage 2, 10× objective. FIG. 11 (c) shows purity of each of the 4 cell populations as indicated by flow cytometry (AT2, HT2-280 expression following CD45/CD90/CD271 depletion; Endothelial, CD144 expression at passage 3; Stromal, CD90 expression at passage 2; AEP, CK5 expression at passage 2). FIG. 11 (d) presents growth characteristics of the endothelial (passage 3), stromal (passage 2), and AEP (passage 2) cultures.

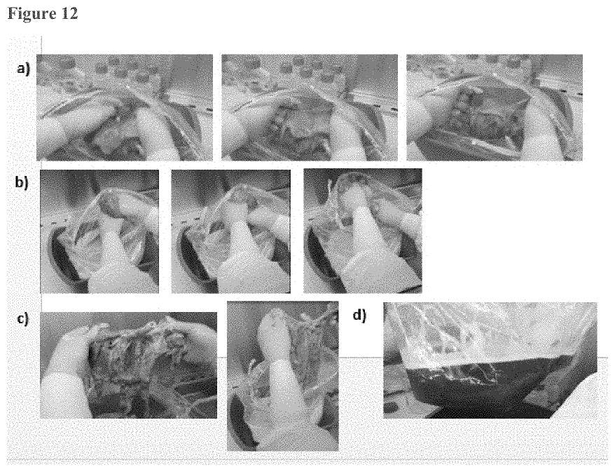

FIG. 12 shows pictures taken of lung crush method being performed on digested lung tissue to demonstrate the process. FIG. 12 (a) shows a series of images demonstrating ripping of the lung pleura to release the digested tissue and cells into a collection bag. FIG. 12 (b) shows a series of images demonstrating squeezing of the lung tissue to release cells into a collection bag. FIG. 12 (c) shows images of two different lungs post-lung crush method demonstrating there is minimal remaining lung tissue after the lung crush dissociation is complete. FIG. 12 (d) shows an image of a resulting cell suspension collected from the lung crush method, demonstrating minimal pieces of intact lung tissue.

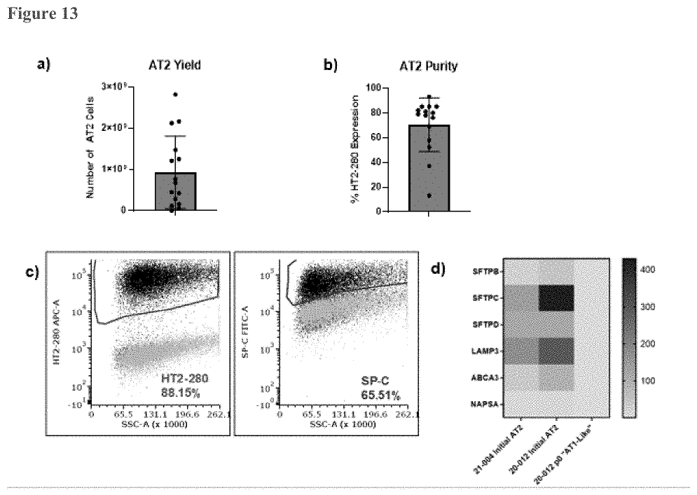

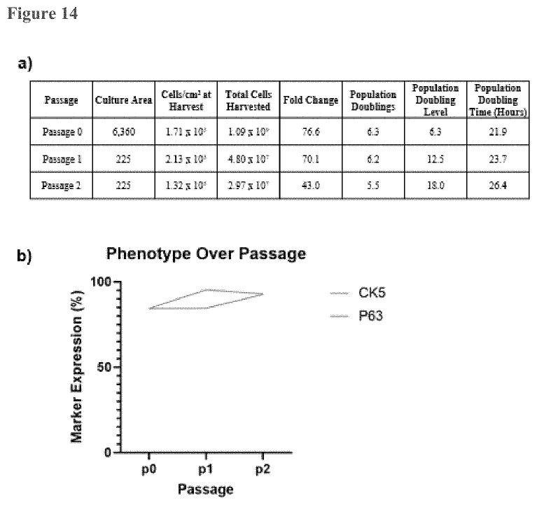

FIG. 13 is a summary of AT2 cell isolation and characterization. FIG. 13 (a) shows AT2 yield (total live cell yield x percent of HT2-280 positive cells) and FIG. 13 (b) shows purity (percent HT2-280 positive cells) across 15 isolations where bilateral lungs were digested and dissociated using lung crush method, purified using magnetic beads to remove CD45, CD271, and CD90 depletion on a CliniMACS instrument using a depletion tubing set, including f runs where a 2-step purification on CliniMACS and MultiMACS was performed as described in FIG. 10, excluding runs with process errors. The mean AT2 yield was 930e6 cells and the mean AT2 purity was 70%. FIG. 13 (c) shows example flow cytometry dot plots of isolated and purified AT2 cells from 1 donor, confirming expected marker expression using HT2-280 and pro-SP-C antibodies (black: target antibody; purple: isotype control). FIG. 13 (d) shows real-time PCR analysis of AT2 gene expression for 2 donors, normalized to alveolar type 1-like (AT1-like) cell gene expression. AT1-like cells were generated by culturing AT2 cells for 7 days in a media intended to promote AT1 conversion. RNA was isolated from the samples using the QIAGEN RNeasy Mini Kit. cDNA was generated and real time PCR was run using probes for genes of interest. These data demonstrate expression of several expected AT2 genes in AT2 cells isolated using the methods described herein, including SFTPB, SFTPC, SFTPD, LAMP3, ABCA3, and NAP SA.

FIG. 14 shows an airway epithelial basal cell isolation and expansion summary from a donor where the airway epithelial basal cells were grown from the selected fraction of a CD45/CD90/CD271 depletion following a lung crush method dissociation as described in Example 2. CD45/CD90/CD271 selected cells were frozen down on the day of isolation, and later thawed and seeded into airway cell media to initiate cultures. Cells were grown for 3 total passages (passage 0 through passage 2). The table in FIG. 14 (a) shows expansion metrics for the airway epithelial basal cells. At every passage, culture area, cells/cm′ at harvest, total cells harvested, fold change, number of population doublings, population doubling level, and population doubling time were collected. More than 1 billion airway basal epithelial basal cells were generated from this donor after just one passage in culture (passage 0). While the size of the subsequent cultures in passage 1 and passage 2 was not maximized, the expansion potential of the basal cells over additional passages was demonstrated. Cell fold change in passage 1 and passage 2 was 70.1 and 43.0, respectively. Population doubling time in passage 1 and passage 2 was 23.7 hrs and 26.4 hrs, respectively. FIG. 14 (b) shows expression of airway epithelial basal cell markers, cytokeratin 5 (ck5) and tumor protein 63 (p63), measured by flow cytometry from passage 0 to passage 2, demonstrating maintenance of basal cell identity over the course of expansion. More than 80% of the population expressed both markers from passage 0 to passage 2. Basal cells from the conducting airway are typically isolated via digesting airway tissue segments, followed by scraping of the airway lumen. An advantage of the method described herein is that the cells can simply be collected from the selected fraction of the purification approach used to isolate AT2 cells, without having to perform a separate cell isolation process and without sacrificing any AT2 cells.

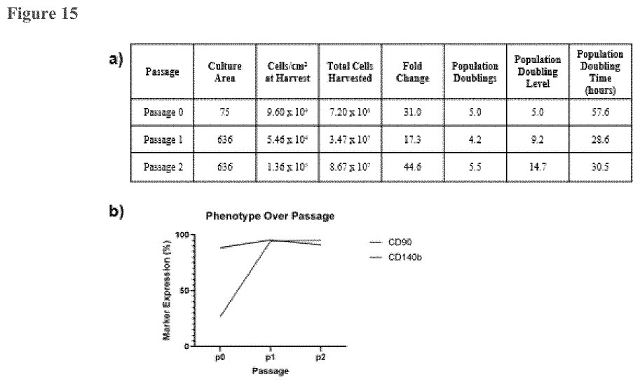

FIG. 15 shows a stromal cell expansion summary from a donor where the stromal cells were grown by seeding post-filtration cells collected from the lung crush method into stromal cell media, as described in Example 3. Post-filtration cells were frozen down on the day of isolation, and later thawed to initiate stromal cultures. Cells were grown for 3 total passages (passage 0 through passage 2). FIG. 15 (a) shows expansion metrics of the stromal cells. For every passage, culture area, cells/cm² at harvest, total cells harvested, fold change, number of population doublings, population doubling level, and population doubling time were collected. The cell yield at passage 0 was 72 million cells, and expansion was continued for 2 additional passages with doubling time decreasing at passages 1 and 2 (28.6 hrs and 30.5 hrs, respectively, compared to 57.6 hrs at passage 0), indicating an increase in cell growth rate. FIG. 15 (b) shows expression of stromal cell markers, CD90 and CD140b, measured by flow cytometry over the course of expansion, demonstrating stromal cell identity was maintained. While CD140b expression was low at passage 0, in passages 1 and 2, more than 80% of cells expressed both CD90 and CD140b. The average post-filtration yield from processing bilateral lungs using lung crush method dissociation is approximately 35 billion cells. Therefore, 20-30 B post-filtration cells can be allocated to AT2 purification, while still leaving excess post-filtration sample to seed stromal cell cultures without sacrificing many of the AT2 or airway epithelial basal cells.

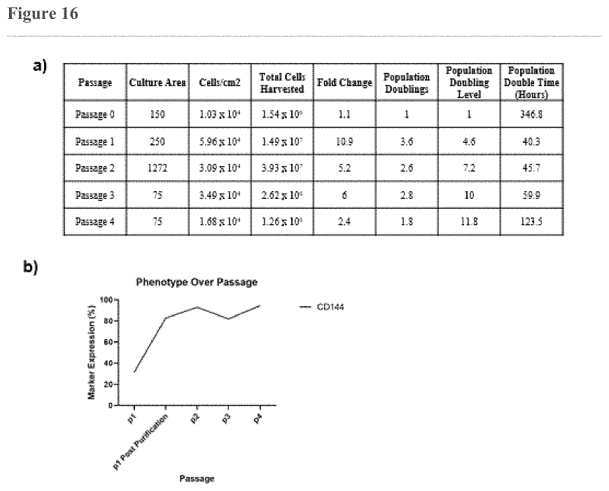

FIG. 16 shows an endothelial cell isolation and expansion summary from a donor where the endothelial cells were grown by seeding post-filtration cells from the lung crush method, as described in Example 4. Post-filtration cells were frozen down on the day of isolation, and later thawed and seeded in endothelial cell expansion media to initiate endothelial cultures. Cells were successfully grown for 5 total passages (passage 0 through passage 4) with a CD31 magnetic bead selection performed after the passage 1 harvest. The table in FIG. 16 (a) shows an expansion summary of the endothelial cells. For every passage, culture area, cells/cm² at harvest, total cells harvested, fold change, number of population doublings, population doubling level, and population doubling time were collected. FIG. 16 (b) shows expression of a marker of endothelial cells, CD144, measured by flow cytometry after every passage, starting at passage 1, including pre- and post-purification. Following the CD31 magnetic bead selection after passage 1, CD144 expression was maintained above 80%.

### Example 6— Isolation of 4 Cell Types from 1 Donor with an Alternative Purification Approach

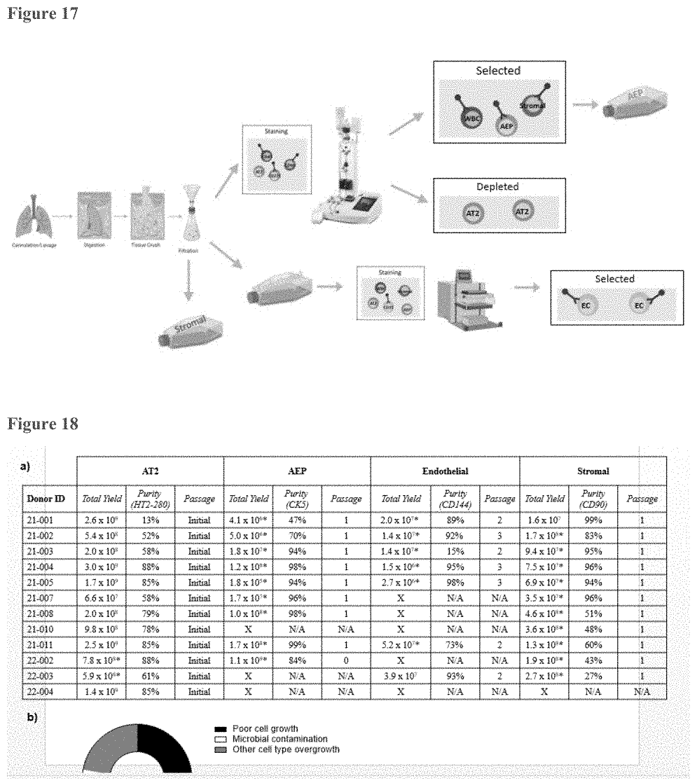

FIG. 17 shows a schematic of a different embodiment of the process to obtain 4 different cell types (AT2 cells, airway epithelial basal cells, stromal cells, and endothelial cells) from one donor. The digestion and tissue dissociation is the same as described in Example 5, but a different purification approach for AT2 and endothelial cells is described in this embodiment. To generate a purified population of AT2 cells, 20-30 B post-filtration cells would be stained with CD45, CD90, and CD271 beads together in one step and run through a depletion column on a CliniMACS instrument without any subsequent purification on a MultiMACS instrument. The depleted cell sample would be designated as the AT2 population. As described in Example 5, to generate a purified population of airway epithelial basal cells, the selected fraction would be seeded in basal cell expansion medium. To generate a purified population of endothelial cells, a portion of the post-filtration sample would be seeded directly into endothelial cell medium on the day of isolation. Cells would then be harvested after 1 passage and purified using a MACS-based CD31 selection approach, as described in Example 4. The endothelial cells would then be put back into culture for subsequent expansion or experimentation. Finally, to generate the stromal cells, a portion of the post-filtration sample would be seeded into stromal cell expansion medium as described in Example 3.

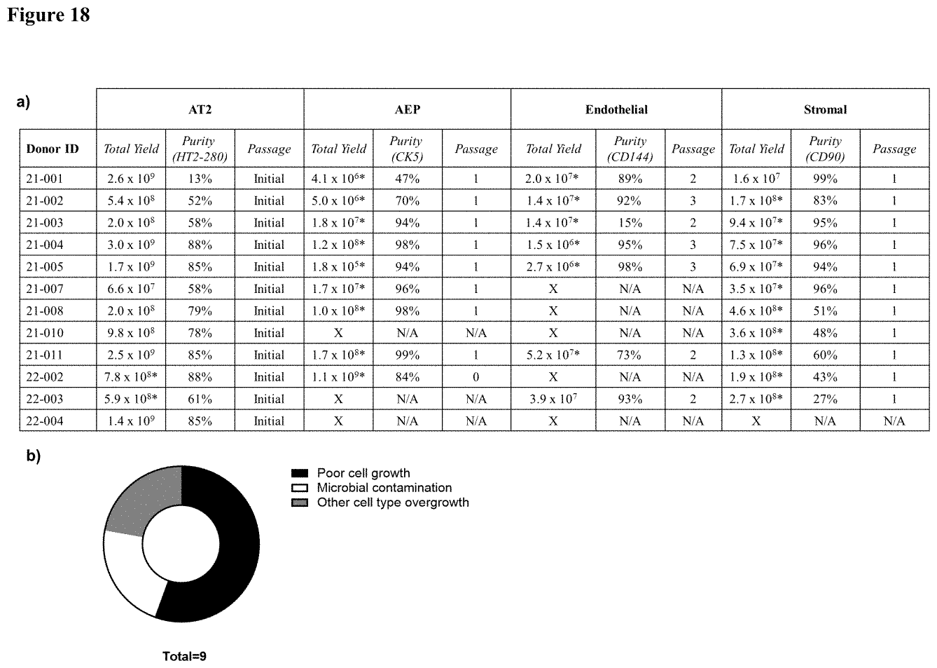

FIG. 18 shows a summary of isolated cells from lungs where lung crush method was used to attempt isolation of 4 different cell types from one donor (AT2: alveolar type 2, AEP: airway epithelial basal, endothelial, and stromal). The table in FIG. 18 (a) shows total cell yield, cell purity, and passage number associated with the reported yield and purity for each cell type. For all cell types in the table, the total yield is the number of live cells counted at harvest from the reported passage, and purity is the percent of the cell type of interest in the total yield measured via flow cytometry. HT2-280 expression was used to determine AT2 cell purity. Ck5 expression was used to determine airway epithelial basal cell purity. CD144 expression was used to determine endothelial cell purity. CD90 expression was used to determine stromal cell purity. Different purification strategies following lung crush method and cell culture media were used for the results presented in this table. An asterisk next to the total yield indicates that the isolation of that cell type was not maximized for that donor and it is therefore predicted that the total cell yield could have been higher. The ‘initial’ passage notation for AT2 cells indicates that the AT2 cells were not expanded prior to analysis of total yield and purity. Cell culture after isolation was used to further purify the other 3 cell types (AEP, endothelial, and stromal), which is why they were included at higher passages. Finally, an X in the total yield column indicates isolation of that cell type was unsuccessful from that donor due to microbial contamination, lack of cell growth, or overgrowth of a different cell type. FIG. 18 (b) shows a breakdown of the reason for each of the unsuccessful isolation attempts noted in FIG. 18 (a).

As used herein, the singular terms “a,” “an,” and “the” may include plural referents unless the context clearly dictates otherwise. Thus, for example, reference to an object may include multiple objects unless the context clearly dictates otherwise.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

While the disclosure has been described with reference to the specific embodiments thereof, it should be understood by those skilled in the art that various changes may be made and equivalents may be substituted without departing from the true spirit and scope of the disclosure as defined by the appended claim(s). In addition, many modifications may be made to adapt a particular situation, material, composition of matter, method, operation or operations, to the objective, spirit and scope of the disclosure. All such modifications are intended to be within the scope of the claim(s) appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations is not a limitation of the disclosure.

1. A method of isolating cells from lung tissue, the method comprising applying mechanical pressure to the lung tissue.

2. The method of claim 1, wherein the application of mechanical pressure results in crushed tissue having no more than 10% by weight of tissue pieces that are 5 mm or larger in size.

3. (canceled)

4. The method of claim 1, wherein the application of mechanical pressure results in crushed tissue having no more than 1% by weight of tissue pieces that are 5 mm or larger in size.

5. The method of claim 1, wherein the lung tissue is crushed.

6. The method of claim 5, wherein the crushing comprises crushing in the hands of a human operator.

7. (canceled)

8. (canceled)

9. (canceled)

10. (canceled)

11. (canceled)

12. (canceled)

13. (canceled)

14. The method of claim 1, wherein the cells are alveolar type II cells.

15. The method of claim 1, wherein the cells are airway epithelial basal cells.

16. The method of claim 1, wherein the cells are stromal cells.

17. The method of claim 1, wherein the cells are endothelial cells.

18. The cells isolated by the method of claim 1.

19. The method of claim 1, wherein the final purified yield of the method is at least 1 billion cells.

20. A method of purifying cell populations of interest from lung tissue isolate, comprising removing white blood cells and at least one other type of cell, wherein the one other type of cell is not a white blood cell.

21. The method of claim 20, wherein antibodies bound to magnetic particles are used to select for and remove the white blood cell and the at least one other type of cell.

22. The method of claim 1, comprising selecting for white blood cells, stromal cells, and airway epithelial basal cells.

23. The method of claim 1, comprising selecting for white blood cells, stromal cells, airway epithelial cells, and endothelial cells.

24. (canceled)

25. (canceled)

26. (canceled)

27. (canceled)

28. A method of isolating cells, comprising:
applying mechanical pressure to lung tissue; and
removing white blood cells and at least one other type of cell from the crushed lung tissue, wherein the one other type of cell is not a white blood cell.

29. (canceled)

30. (canceled)

31. (canceled)

32. A method of forming an engineered lung structure, the method comprising seeding a lung scaffold with cells obtained from a method of claim 1.

33. The method of claim 32, wherein the cells from the method of claim 1 are separated from other cells after a step of using one or more CD markers to remove white blood cells and at least one other type of cell from the cell suspension.

34. (canceled)

35. (canceled)

36. The method of claim 28, wherein the seeded cells are alveolar Type II cells.

37. The method of claim 28, wherein the seeded cells are airway basal cells.

38. The method of claim 28, wherein the seeded cells are lung stromal cells.

39. The method of claim 28, wherein the seeded cells are lung endothelial cells.
